(19) United States
(12) Patent Application Publication
(10) Pub. No.: US 2008/0165922 A1
(43) Pub. Date: Jul. 10, 2008

(54) LAMINATED CT COLLIMATOR AND METHOD OF MAKING SAME

(76) Inventors: Brian David Yanoff, Niskayuna, NY (US); Jonathan D. Short, Saratoga Springs, NY (US); Richard A. Thompson, Clifton Park, NY (US); Bruce Campbell Amm, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/621,458

(22) Filed: Jan. 9, 2007

(51) Int. Cl. A61B 6/00; G21K 1/02

(52) U.S. Cl. 378/19; 378/147

(57) ABSTRACT

A CT collimator includes a first radiation absorbent lamination having a plurality of apertures formed therethrough. Each aperture formed through the first radiation absorbent lamination is aligned with a respective axis formed between a corresponding pixellating element and an x-ray emission source. The collimator includes a second radiation absorbent lamination having a plurality of apertures formed therethrough, each aperture formed through the second radiation absorbent lamination aligned with the respective axis formed between a corresponding pixellating element and the x-ray emission source. A spacer is positioned between the first and second radiation absorbent laminations.

### BACKGROUND OF THE INVENTION

The present invention relates generally to diagnostic imaging and, more particularly, to a laminated CT detector collimator and methods of making same.

Typically, in computed tomography (CT) imaging systems, an x-ray source emits a fan-shaped beam toward a subject or object, such as a patient or a piece of luggage. Hereinafter, the terms “subject” and “object” shall include anything capable of being imaged. The beam, after being attenuated by the subject, impinges upon an array of radiation detectors. The intensity of the attenuated beam radiation received at the detector array is typically dependent upon the attenuation of the x-ray beam by the subject. Each detector element of the detector array produces a separate electrical signal indicative of the attenuated beam received by each detector element. The electrical signals are transmitted to a data processing system for analysis which ultimately produces an image.

Generally, the x-ray source and the detector array are rotated about a gantry within an imaging plane and around the subject. X-ray sources typically include x-ray tubes, which emit the x-ray beam at a focal point. X-ray detectors typically include a collimator having a plurality of collimator plates for collimating x-ray beams received at the detector, a scintillator for converting x-rays to light energy adjacent the collimator, and photodiodes for receiving the light energy from the adjacent scintillator and producing electrical signals therefrom.

An x-ray detector may, instead of using a scintillating device, include an energy discriminating detector having a direct conversion material capable of x-ray counting and capable of providing a measurement of the energy level of each x-ray detected. A laminated collimator as described herein is equally applicable to use with an energy discriminating device or other detector using pixellated elements.

Typically, each scintillator of a scintillator array converts x-rays to light energy. Each scintillator discharges light energy to a photodiode adjacent thereto. Each photodiode detects the light energy and generates a corresponding electrical signal. The outputs of the photodiodes are then transmitted to the data processing system for image reconstruction.

Image quality can be directly associated with the degree of alignment between the components of the detector. “Cross-talk” between detector cells of a CT detector is common and to some degree is affected by the alignment, or lack thereof, of the detector components. In this regard, cross-talk is typically higher when the components of the CT detector are misaligned.

Cross-talk is generally defined as the communication of data between adjacent cells of a CT detector. Generally, cross-talk is sought to be reduced because cross-talk leads to artifact presence in the final reconstructed CT image and contributes to poor spatial resolution. Different types of cross-talk may result within a single CT detector. Cross-talk can occur as light from one cell is passed to another through a contiguous layer between the photodiode layer and the scintillator. Electrical cross-talk can occur from unwanted communication between photodiodes. Optical cross-talk may occur through the transmission of light through the reflectors that surround the scintillators. X-ray cross-talk may occur due to x-ray scattering between scintillator cells.

In an effort to reduce cross-talk, plates or layers of a collimator may be aligned with the cells of the scintillator arrays. The alignment of the cells of the scintillator arrays and the plates of the collimator can be a time consuming and labor intensive process. A collimator is typically fabricated using approximately 1000 collimating plates that are inserted between a set of rails. The rails typically have combs attached thereto, each comb having a plurality of teeth that are constructed to hold the collimating plates. Typically the rails are aligned to very exacting tolerances such that the teeth of the combs are positioned to receive the collimating plates, and, when inserted into the teeth, provide a collimating effect to the pixellating elements. Further, the physical placement or alignment of the collimator to the scintillator array is particularly susceptible to misalignment stack-up. That is, one of the scintillator-collimator assemblies, if unaligned, can detrimentally affect the alignment of adjacent assemblies. Simply, if one collimator-scintillator array combination is misaligned, all subsequently positioned collimator-scintillator array combinations will be misaligned absent implementation of corrective measures. Further, such assemblies require adjusting several detectors when only one of the detectors is misaligned. The overall process can be costly and time-consuming.

Mechanical deflection of the plates can occur due to G loading induced by rotation of the gantry at high gantry speeds. If a CT system is calibrated at, for instance, one speed and image data is acquired at, for instance, a second speed, then mechanical deflection of the collimator plates may be different for the two gantry speeds. Such mechanical deflection may induce image artifacts in resulting images. Additionally, Z-axis coverage of patients has increased in recent years and, and increased Z-axis coverage requires, likewise, proportionately longer collimator plates. Accordingly, the collimator plates are thus increasingly susceptible to mechanical deflection and the image quality problems resulting therefrom.

Therefore, it would be desirable to design a method and apparatus for the fabrication of a low-cost collimator and a scintillator module to thereby reduce cross-talk and increase mechanical stability thereof.

### BRIEF DESCRIPTION OF THE INVENTION

The present invention is directed to an apparatus that overcomes the aforementioned drawbacks. The CT detector includes a plurality of pixellated elements and a laminated collimator. Laminations within the collimator are separated by a spacer material and have apertures aligned between a respective pixellating element and an x-ray source.

According to one aspect of the present invention, a CT collimator includes a first radiation absorbent lamination having a plurality of apertures formed therethrough. Each aperture formed through the first radiation absorbent lamination is aligned with a respective axis formed between a corresponding pixellating element and an x-ray emission source. The collimator includes a second radiation absorbent lamination having a plurality of apertures formed therethrough, each aperture formed through the second radiation absorbent lamination aligned with the respective axis formed between a corresponding pixellating element and the x-ray emission source. A spacer is positioned between the first and second radiation absorbent laminations.

According to another aspect of the present invention, a method of fabricating a CT detector, includes providing a detector having a plurality of pixellated elements and coupling a multi-laminate collimator to the detector. The multi-laminate collimator includes at least two layers of material substantially impermeable to radiation. The method includes positioning an insert between the at least two layers, and aligning the collimator such that a plurality of x-ray passageways within the collimator are aligned between the plurality of pixellated elements and an x-ray emission source in a 1:1 correspondence.

In accordance with another aspect of the present invention, a CT system includes a rotatable gantry having an opening to receive an object to be scanned, a high frequency electromagnetic energy projection source configured to project a high frequency electromagnetic energy beam toward the object, and a detector array having a plurality of pixellated cells wherein each cell is configured to detect high frequency electromagnetic energy passing through the object. A radiation filter is configured to absorb high frequency electromagnetic energy directed toward a space between adjacent pixellated cells, wherein the radiation filter includes a pair of perforated screens separated at least by a spacer material. A photodiode array is optically coupled to the scintillator array and includes a plurality of photodiodes configured to detect light output from a corresponding scintillator cell. A data acquisition system (DAS) is connected to the photodiode array and configured to receive the photodiode outputs. An image reconstructor connected to the DAS and configured to reconstruct an image of the object from the photodiode outputs received by the DAS.

Various other features and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a pictorial view of a CT imaging system according to the present invention.

FIG. 2 is a block schematic diagram of the system illustrated in FIG. 1.

FIG. 3 is a perspective view of one embodiment of a CT system detector array.

FIG. 4 is a perspective view of one embodiment of a detector of the detector array shown in FIG. 3.

FIG. 5 is a cross-sectional view of a portion of a scintillator pack and laminated collimator having a spacer according to an embodiment of the present invention.

FIG. 6 is a perspective view of a collimator having laminates positioned between assembly pins according to an embodiment of the present invention.

FIG. 7 is a pictorial view of an apparatus for slicing structural foam according to an embodiment of the present invention.

FIG. 8 is a cross-sectional view of a portion of a scintillator pack and laminated collimator having an encapsulating material spacer according to an embodiment of the present invention.

FIG. 9 is a cross-sectional view of a portion of a laminated collimator having a structural foam spacer according to an embodiment of the present invention.

FIG. 10 is a cross-sectional view of a portion of a laminated collimator having a plurality of spacers according to an embodiment of the present invention.

FIG. 11 is a pictorial view of a CT system for use with a non-invasive package inspection system according to an embodiment of the present invention.

### DETAILED DESCRIPTION OF THE PREFERRED EMBODIMENT

The operating environment of the present invention is described with respect to a sixty-four-slice computed tomography (CT) system. However, it will be appreciated by those skilled in the art that the present invention is equally applicable for use with other multi-slice configurations. Moreover, the present invention will be described with respect to the detection and conversion of x-rays. However, one skilled in the art will further appreciate that the present invention is equally applicable for the detection and conversion of other high frequency electromagnetic energy. The present invention will be described with respect to a “third generation” CT scanner, but is equally applicable with other CT systems.

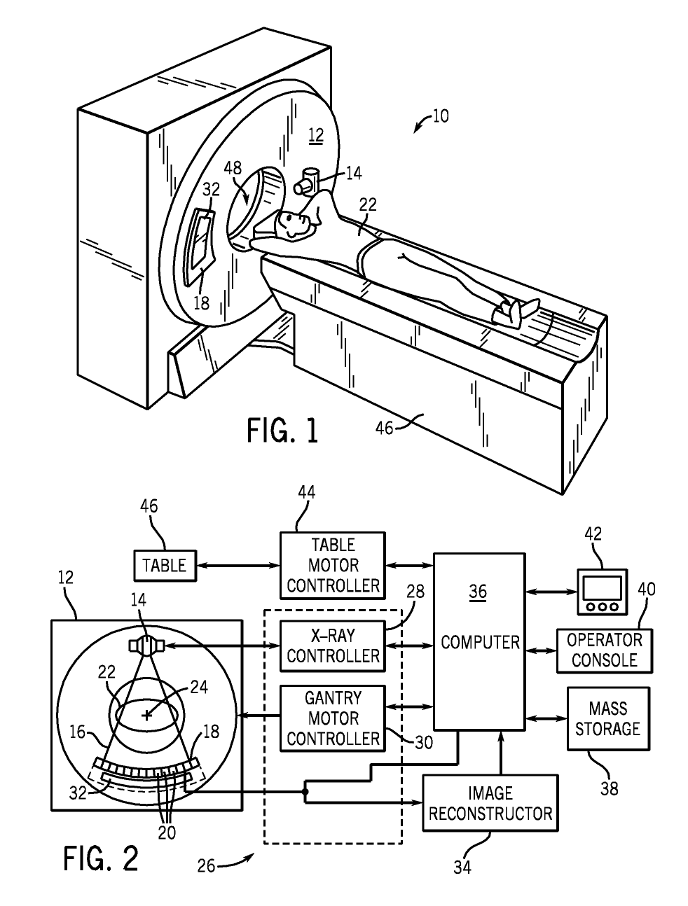

Referring to FIGS. 1 and 2, a computed tomography (CT) imaging system 10 is shown as including a gantry 12 representative of a “third generation” CT scanner. Gantry 12 has an x-ray source 14 that projects a beam of x-rays 16 toward a detector assembly or collimator 18 on the opposite side of the gantry 12. Detector assembly 18 is formed by a plurality of detectors 20 and data acquisition systems (DAS) 32. The plurality of detectors 20 sense the projected x-rays that pass through a medical patient 22, and DAS 32 converts the data to digital signals for subsequent processing. Each detector 20 produces an analog electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14 and a gantry motor controller 30 that controls the rotational speed and position of gantry 12. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves portions of patient 22 through a gantry opening 48.

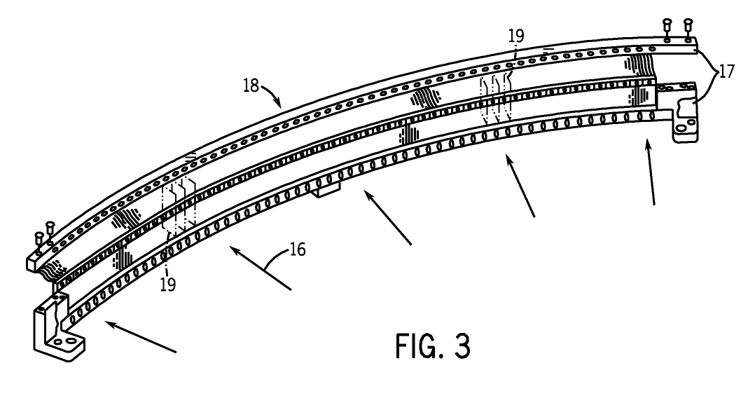

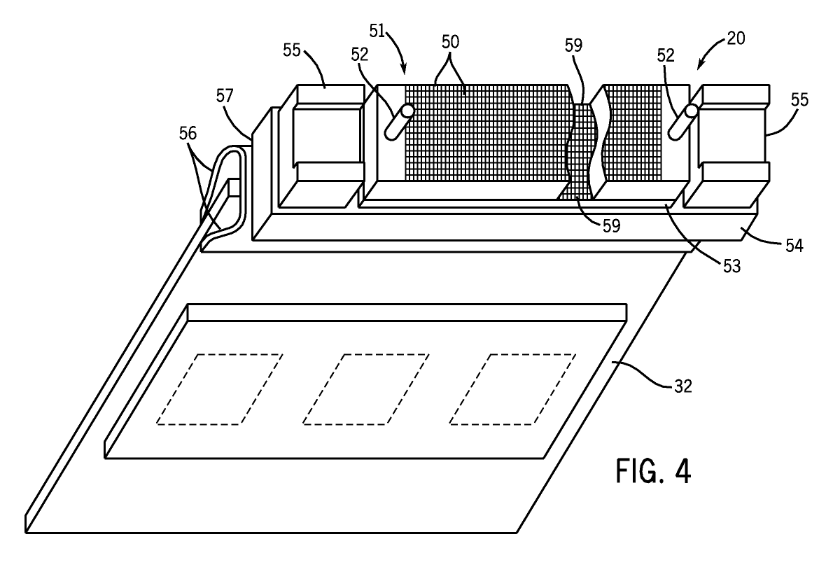

As shown in FIGS. 3 and 4, detector assembly 18 includes a plurality of detectors 20 and DAS 32, with each detector 20 including a number of detector elements 50 arranged in pack 51. Rails 17 of the detector assembly 18 collimator have collimating blades or plates 19 placed therebetween. Detector assembly 18 is positioned to collimate x-rays 16 before such beams impinge upon the detector 20. In one embodiment, shown in FIG. 3, detector assembly 18 includes 57 detectors 20, each detector 20 having an array size of 64×16 of pixel elements 50. As a result, detector assembly 18 has 64 rows and 912 columns (16×57 detectors) which allows 64 simultaneous slices of data to be collected with each rotation of gantry 12.

Detectors 20 include pins 52 positioned within pack 51 relative to detector elements 50. Pack 51 is positioned on diode array 53 having a plurality of diodes 59. Diode array 53 is in turn positioned on multi-layer substrate 54. Spacers 55 are positioned on multi-layer substrate 54. Detector elements 50 are optically coupled to diode array 53, and diode array 53 is in turn electrically coupled to multi-layer substrate 54. Flex circuits 56 are attached to face 57 of multi-layer substrate 54 and to DAS 32. Detectors 20 are positioned within detector assembly 18 by use of pins 52.

In operation, x-rays impinging within detector elements 50 generate photons which traverse pack 51, thereby generating an analog signal which is detected on a diode 58 within diode array 53. The analog signal generated is carried through multi-layer substrate 54, through one of flex circuits 56, to DAS 32 wherein the analog signal is converted to a digital signal.

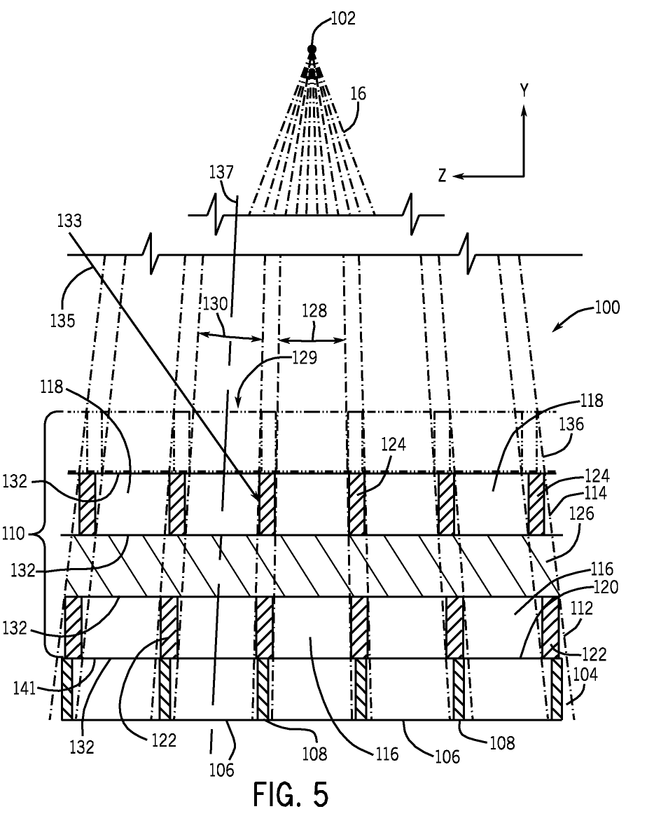

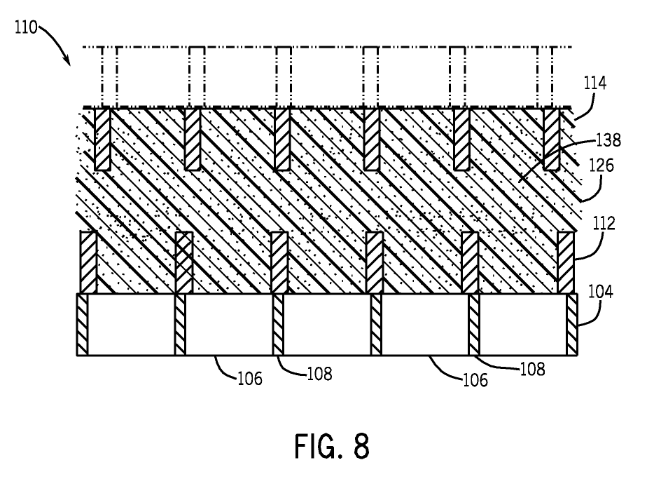

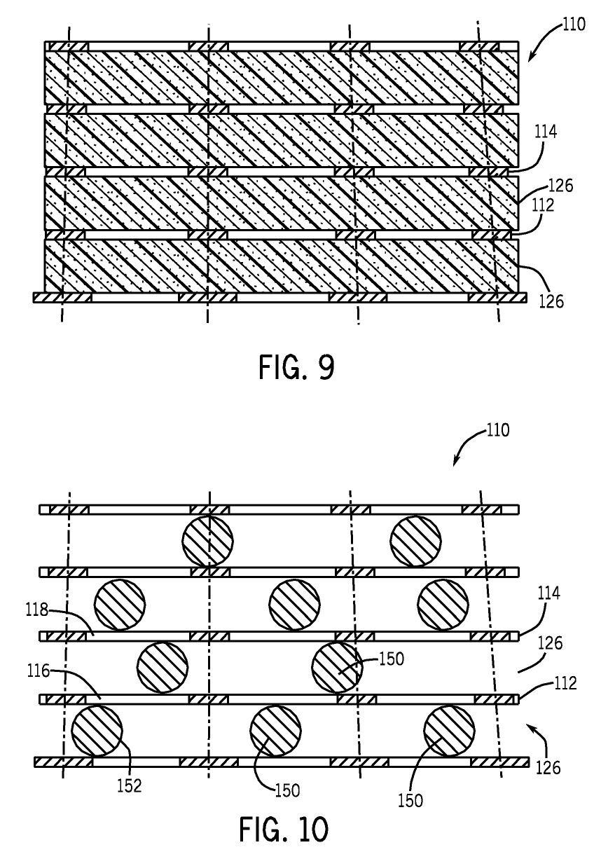

FIGS. 5 and 8 illustrate a portion of a CT detector 100 according to an embodiment of the present invention. For illustrative purposes, the thicknesses of laminates 112, 114, and 136 in FIGS. 5 and 8 are enlarged to better show the passage of x-rays 16 therethrough. However, the thickness of laminates 112, 114, and 136 shown in FIGS. 9 and 10 best illustrate the proportionality thereof with respect to the CT detector 100 as a whole.

FIG. 5 is a portion of a CT detector according to an embodiment of the present invention. CT detector 100 includes a scintillator pack 104 and a collimator 110. CT detector 100 is oriented in the X-Z plane of a rotating gantry coordinate system. and positioned such that x-rays 16 emitting from an x-ray focal spot 102 of an x-ray tube, such as x-ray tube 14 of FIG. 1, are directed toward scintillator pack 104. Scintillator pack 104 includes pixels, or scintillator elements, 106 separated by reflectors 108. While the portion of the CT detector 100 of FIG. 5 shows five pixels 106 with a corresponding portion of the collimator 110, one skilled in the art will recognize that the number of pixels 106 in CT detector 100 may include more than the five pixels 106 shown.

A first laminate, or screen, 112 of collimator 110 is positioned proximate to scintillator pack 104. Laminate 112 is perforated such that each perforation, or aperture, 116 formed therein is sized and positioned to allow x-rays 16 to pass therethrough to impinge on an upper surface 120 of a corresponding pixel 106. In this manner, each perforation 116 is substantially aligned with an axis 137 formed between the corresponding pixel 106 and the focal spot 102. The perforations 116 of laminate 112 are further sized and positioned such that structural material 122 of laminate 112 is positioned to obstruct x-rays 16 that emit from focal spot 102 toward reflectors 108.

A second laminate, or screen, 114 of collimator 110 is positioned proximate to laminate 112 is perforated such that each perforation, or aperture 118 formed therein is sized and positioned to allow x-rays 16 to pass therethrough to impinge on an upper surface 120 of a corresponding pixel 106. In this manner, each perforation 118 is substantially aligned with an axis, one of which is illustrated as axis 137, formed between the corresponding pixel 106 and the focal spot 102. Accordingly, each pair of perforations 116 and 118 corresponding to each pixel 106 forms a hole, or opening, 129 through collimator 110 that is substantially aligned with a respective axis 137 formed between the corresponding pixel 106 and the focal spot 102. The perforations 118 of laminate 114 are further sized and positioned such that structural material 124 of laminate 114 is positioned to obstruct x-rays 16 that emit from focal spot 102 toward reflectors 108.

A fanout angle 128 is formed between one pixel 106 and focal spot 102 of one axis 139 and another fanout angle 130 is formed between another pixel 106 and focal spot 102 of another axis 137. A pattern of the perforations 116 of the first laminate 112 may be distinct from a pattern of the perforations 118 of the second laminate 114. Accordingly, in one embodiment, perforations 116 have a larger opening and are positioned closer together than respective perforations 118. In another embodiment, perforations 116 are positioned further apart than respective perforations 118 but have an opening substantially similar to an opening of respective perforations 118. It is contemplated, however, that the patterns of respective perforations 116, 118 are substantially similar and are sized and positioned according to the fanout as defined by, for instance, fanout angles 128 and 130. Additionally, according to the fanout angle 128, perforations 116 in laminate 112 may have different sizes and spacings with respect to each other. Similarly, according to the fanout angle 130, perforations 118 in laminate 118 may have different sizes and spacings with respect to each other.

Laminae 112, 114 comprise a high density material, such as tungsten or the like. Accordingly, laminae 112, 114 are substantially impermeable to and substantially attenuate x-rays 16 that would otherwise impinge on the region of reflectors 108 in scintillator pack 104. It is contemplated that the perforations 116, 118 therein are fabricated by etching, drilling, molding, or the like.

Still referring to FIG. 5, a spacer, or laminate, 126 is positioned between laminae 112, 114. In one embodiment, spacer 126 is made of a pre-cured closed-cell structural foam such as Rohacell™, graphite sheets, and the like, such that spacer 126 is substantially transparent to x-rays 16. Rohacell™ is available from Degussa AG of Dusseldorf, Germany. In another embodiment discussed below with respect to FIG. 8, spacer 126 is an encapsulating material and is cured in situ. Spacer 126 structurally supports laminae 112, 114 and increases G load tolerance of collimator 110 during gantry rotation. An adhesive 141 inserted at surfaces 132 between each layer 104, 112, 114, 126 of CT detector 100 binds layers 104, 112, 114, 126 together and contributes to structural integrity of CT detector 100.

In operation, collimator 110 substantially attenuates x-rays 16 that emit from focal spot 102 from impinging on reflectors 108. Collimator 110 also collimates x-rays that emit from a secondary emission point 133 within, for instance, patient 22 of FIGS. 1 and 2, and travel along path 135. Accordingly, collimator 110 substantially allows x-rays 16 that emit from focal spot 102 to impinge on pixels 106, and x-rays 133 that derive from secondary emissions are substantially attenuated.

Referring still to FIG. 5, a collimator 110 having laminate 112, spacer 126, and laminate 114 is illustrated. In one embodiment, additional laminae, not shown, may be added to achieve a collimator depth of, for instance, 7-8 mm or greater, depending on a desired aspect ratio between openings formed by 116, 118, and a total collimator 110 stack height. One skilled in the art will recognize that laminae may be stacked directly on one another to form a multi-laminate attenuating material and not having a spacer therebetween, or may stack several laminae together before placing a spacer therebetween, so long as the geometric spacing between laminae is taken into account to accommodate, as an example, fanout angles 128, 130. For instance, in an alternate embodiment laminate 136 is positioned directly in contact with, and attached to, laminate 114. Such may be advantageous to allow the use of much thinner laminate materials when, for instance, very precise features are desired, and such precision is more difficult or costly to obtain when using thicker laminae.

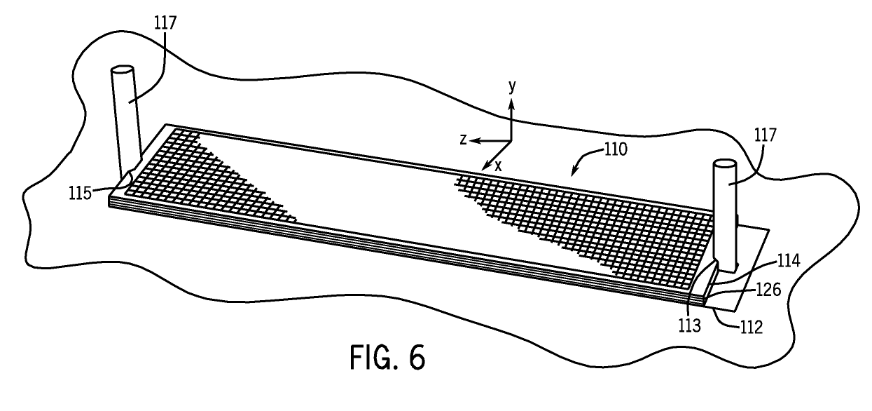

Furthermore, while CT detector 100 is illustrated in FIG. 5 in a two-dimensional layout in the Y-Z plane, one skilled in the art will recognize that the pattern of fanout angles as illustrated by fanout angles 128, 130, may fanout in a similar fashion in the X-Y plane as well, thus forming a three-dimensional collimator with fanout angles of laminae projecting in both dimensions. FIG. 6 illustrates a stack of laminae 112, 114 having spacer 126 positioned therebetween. FIG. 6 illustrates the three-dimensional fanout requirements of collimator 110 and the corresponding fanout angles which may be achieved in both the X-Y plane and the Y-Z plane to achieve the three-dimensional fanout effect.

Referring again to FIG. 6, laminae 112, 114 may each have notches 113, 115 positioned therein. The notches 113, 115 positioned in each laminate 112, 114 are positioned such that the notches 113, 115 align vertically when assembled as a unit against, for instance, pins 117. Such alignment enables simple construction of collimator 110 and enable a quick visual check of the assembled unit to confirm proper alignment of laminae 112, 114 with respect to each other. One skilled in the art will recognize that notches 113, 115 may instead include protruding alignment tabs for construction of collimator 110.

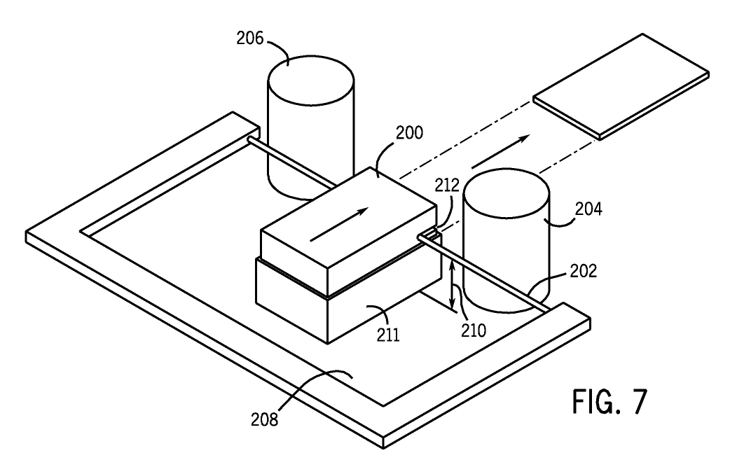

Referring to FIG. 7, structural foam 200 such as Rohacell™ and the like may be cut into thin sheets using a hot, 0.014″ thick wire 202, such as inconel, stretched taut between two ceramic cylinders 204, 206 and positioned at a desired height 210 above the flat surface 208. A sheet of structural foam 200 is placed on flat surface 208 and held thereto while being fed transverse to the wire 202, thereby slicing a thin foam piece 212 to be used for spacer 126. Structural foam 200 may be placed on feed material 211 and traversed through wire 202 to slice thin sheets of structural foam 200.

Referring now to FIG. 8, as described above, in one embodiment of the present invention, spacer 126 may be an encapsulating material 138 positioned within collimator 110. Accordingly, laminae 112, 114 are positioned such that gap 140 is formed therein. Encapsulating material 138, such as epoxy or structural foam, is positioned within gap 140 and cured in situ. Encapsulating material 138 is selected of materials that are substantially transparent to the passage of x-rays. Encapsulating material 138 may be, for instance, an uncured foam or epoxy that is caused to be injected or otherwise flowed into gap 140 and allowed to cure. Furthermore, in order to reduce the density of the encapsulating material 138, a low density filler such as hollow glass micro-beads may be mixed therewith, the low density filler preferably having an average density less than that of encapsulating material 138.

FIG. 9 shows collimator 110 according to an embodiment of the present invention. Laminae 112, 114 are positioned between spacers 126 and best illustrate the proportionality of collimator 110 as a whole.

FIG. 10 shows a spacer 126 of CT detector 100 according to another embodiment of the present inventions. A plurality of thin tubes 150 may be positioned substantially between laminae 112 and 114 such that x-rays passing through openings 116, 118 are randomly obstructed, thereby avoiding a pattern of obstructions that may create image artifacts. Thin tubes 150 may have a circular 152 or other shaped cross-section such as for instance rectangular or hexagonal and may also be randomly oriented between laminae 112 and 114.

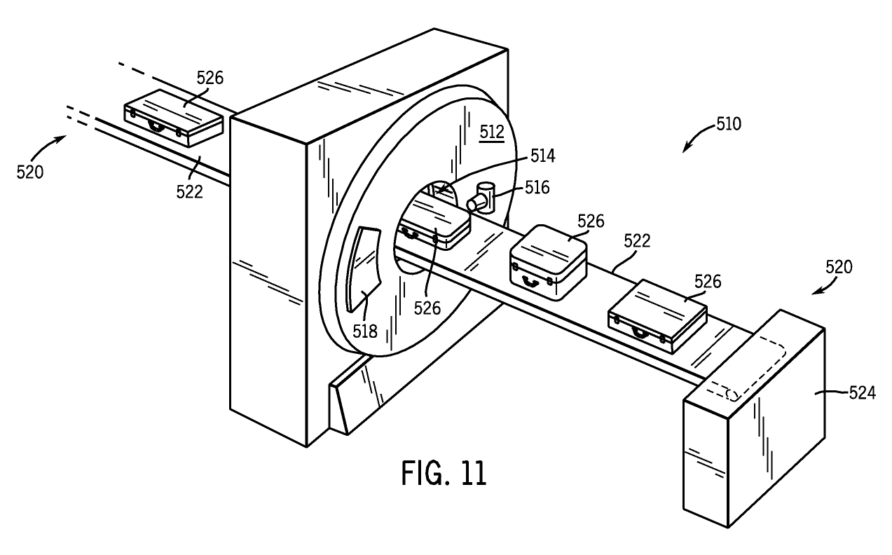

FIG. 11 is a pictorial view of a CT system for use with a non-invasive package inspection system. Package/baggage inspection system 510 includes a rotatable gantry 512 having an opening 514 therein through which packages or pieces of baggage may pass. The rotatable gantry 512 houses a high frequency electromagnetic energy source 516 according to an embodiment of the present invention, as well as a detector assembly 518 having scintillator arrays comprised of scintillator cells. A conveyor system 520 is also provided and includes a conveyor belt 522 supported by structure 524 to automatically and continuously pass packages or baggage pieces 526 through opening 514 to be scanned. Objects 526 are fed through opening 514 by conveyor belt 522, imaging data is then acquired, and the conveyor belt 522 removes the packages 526 from opening 514 in a controlled and continuous manner. As a result, postal inspectors, baggage handlers, and other security personnel may non-invasively inspect the contents of packages 526 for explosives, knives, guns, contraband, etc. Additionally, such systems may be used in industrial applications for non-destructive evaluation of parts and assemblies.

Therefore, according to one embodiment of the present invention, a CT collimator includes a first radiation absorbent lamination having a plurality of apertures formed therethrough. Each aperture formed through the first radiation absorbent lamination is aligned with a respective axis formed between a corresponding pixellating element and an x-ray emission source. The collimator includes a second radiation absorbent lamination having a plurality of apertures formed therethrough, each aperture formed through the second radiation absorbent lamination aligned with the respective axis formed between a corresponding pixellating element and the x-ray emission source. A spacer is positioned between the first and second radiation absorbent laminations.

According to another embodiment of the present invention, a method of fabricating a CT detector, includes providing a detector having a plurality of pixellated elements and coupling a multi-laminate collimator to the detector. The multi-laminate collimator includes at least two layers of material substantially impermeable to radiation. The method includes positioning an insert between the at least two layers, and aligning the collimator such that a plurality of x-ray passageways within the collimator are aligned between the plurality of pixellated elements and an x-ray emission source in a 1:1 correspondence.

In accordance with another embodiment of the present invention, a CT system includes a rotatable gantry having an opening to receive an object to be scanned, a high frequency electromagnetic energy projection source configured to project a high frequency electromagnetic energy beam toward the object, and a detector array having a plurality of pixellated cells wherein each cell is configured to detect high frequency electromagnetic energy passing through the object. A radiation filter is configured to absorb high frequency electromagnetic energy directed toward a space between adjacent pixellated cells, wherein the radiation filter includes a pair of perforated screens separated at least by a spacer material. A photodiode array is optically coupled to the scintillator array and includes a plurality of photodiodes configured to detect light output from a corresponding scintillator cell. A data acquisition system (DAS) is connected to the photodiode array and configured to receive the photodiode outputs. An image reconstructor connected to the DAS and configured to reconstruct an image of the object from the photodiode outputs received by the DAS.

The present invention has been described in terms of the preferred embodiment, and it is recognized that equivalents, alternatives, and modifications, aside from those expressly stated, are possible and within the scope of the appending claims.

1. (canceled)

2. (canceled)

3. A CT collimator positioned proximate to a CT detector, the CT collimator comprising:
a first radiation absorbent lamination having a plurality of apertures formed therethrough, each aperture formed through the first radiation absorbent lamination aligned with a respective axis formed between a corresponding pixellating element and an x-ray emission source;
a second radiation absorbent lamination having a plurality of apertures formed therethrough, each aperture formed through the second radiation absorbent lamination aligned with the respective axis formed between a corresponding pixellating element and the x-ray emission source: and a spacer substantially transparent to x-rays positioned between the first and second radiation absorbent laminations; and wherein the spacer comprises one of a foam, a graphite sheet, an epoxy, a fiber, and a tube.

4. The CT collimator of claim 3 wherein the epoxy has a filler material dispersed therein, the filler material having an average density less than that of the epoxy.

5. The CT collimator of claim 3 wherein the tube has a circular cross-section.

6. The CT collimator of claim 3 wherein the foam is cured in situ.

7. (canceled)

8. (canceled)

9. (canceled)

10. (canceled)

11. A CT collimator positioned proximate to a CT detector, the CT collimator comprising:
a first radiation absorbent lamination having a plurality of apertures formed therethrough, each aperture formed through the first radiation absorbent lamination aligned with a respective axis formed between a corresponding pixellating element and an x-ray emission source;
a second radiation absorbent lamination having a plurality of apertures formed therethrough, each aperture formed through the second radiation absorbent lamination aligned with the respective axis formed between a corresponding pixellating element and the x-ray emission source;
a spacer substantially transparent to x-rays positioned between the first and second radiation absorbent laminations; and wherein the spacer is a sheet of material having a plurality of apertures formed therethrough.

12. (canceled)

13. (canceled)

14. (canceled)

15. (canceled)

16. A method of fabricating a CT detector, the method comprising:
providing a detector having a plurality of pixellated elements;
coupling a multi-laminate collimator to the detector, the multi-laminate collimator comprising at least two layers of material substantially impermeable to radiation;
positioning an insert between the at least two layers;
aligning the collimator such that a plurality of x-ray passageways within the collimator are aligned between the plurality of pixellated elements and an x-ray emission source in a 1:1 correspondence; and
wherein positioning comprises injecting an uncured foam into a space between the at least two layers.

17. A method of fabricating a CT detector, the method comprising:
providing a detector having a plurality of pixellated elements;
coupling a multi-laminate collimator to the detector, the multi-laminate collimator comprising at least two layers of material substantially impermeable to radiation;
positioning an insert substantially transparent to x-rays between the at least two layers:
aligning the collimator such that a plurality of x-ray passageways within the collimator are aligned between the plurality of pixellated elements and an x-ray emission source in a 1:1 correspondence; and
wherein positioning comprises inserting a structural foam into a space between the at least two layers.

18. The method of claim 17 further comprising slicing the structural foam using a hot wire cutter.

19. A method of fabricating a CT detector, the method comprising:
providing a detector having a plurality of pixellated elements;
coupling a multi-laminate collimator to the detector, the multi-laminate collimator comprising at least two layers of material substantially impermeable to radiation;
positioning an insert substantially transparent to x-rays between the at least two layers:
aligning the collimator such that a plurality of x-ray passageways within the collimator are aligned between the plurality of pixellated elements and an x-ray emission source in a 1:1 correspondence; and
wherein positioning comprises inserting one of a graphite sheet, an epoxy, a fiber, and a tube into a space between the at least two layers.

20. (canceled)

21. (canceled)

22. A CT system comprising:
a rotatable gantry having an opening to receive an object to be scanned;
a high frequency electromagnetic energy projection source configured to project a high frequency electromagnetic energy beam toward the object;
a detector array having a plurality of pixellated cells wherein each cell is configured to detect high frequency electromagnetic energy passing through the object;
a radiation filter configured to absorb high frequency electromagnetic energy directed toward a space between adjacent pixellated cells, wherein the radiation filter comprises a pair of perforated screens separated at least by a spacer material substantially transparent to x-rays;
a photodiode array optically coupled to the scintillator array and comprising a plurality of photodiodes configured to detect light output from a corresponding scintillator cell;
a data acquisition system (DAS) connected to the photodiode array and configured to receive the photodiode outputs;
an image reconstructor connected to the DAS and configured to reconstruct an image of the object from the photodiode outputs received by the DAS; and
wherein the spacer is one of a foam, a graphite sheet, an epoxy, a fiber, and a tube.

23. (canceled)
